(19) United States
(12) Patent Application Publication
(10) Pub. No.: US 2011/0217337 A1
(43) Pub. Date: Sep. 8, 2011

(54) BIOACTIVE AGENT FOR BONE TISSUE ENGINEERING

(75) Inventors: Cato T. Laurencin, Avon, CT (US); Wai Hong Lo, Plainville, CT (US)

(73) Assignee: UNIVERSITY OF CONNECTICUT, Farmington, CT (US)

(21) Appl. No.: 13/038,792

(22) Filed: Mar. 2, 2011

Related U.S. Application Data
(60) Provisional application No. 61/339,261, filed on Mar. 2, 2010.

(51) Int. Cl. A61K 9/00; A61K 31/7076; A61K 35/12; A61K 38/18; A61P 19/00; A61P 43/00; C12N 11/08

(52) U.S. Cl. 424/400; 514/47; 424/93.7; 514/8.8; 435/180

(57) ABSTRACT

Inventors have discovered a method for inducing bone formation in a patient in need thereof comprising administering an effective amount of benzoyladenosine-3′,5′-cyclic monophosphate (6-Bnz-cAMP) to the patient. Systems for bone tissue engineering, comprising a polymer-based scaffold, such as a PLAGA scaffold and an effective amount of 6-Bnz-cAMP are also disclosed herein.

### CROSS REFERENCE TO RELATED APPLICATION

This application claims priority from U.S. Provisional Application No. 61/339,261, filed Mar. 2, 2010, which is hereby incorporated by reference in its entirety.

### FIELD OF DISCLOSURE

The present disclosure is in the field of tissue engineering and, more specifically, in the field of methods, systems, and agents useful in bone regeneration and repair. Inventors have found N⁶-Benzoyladenosine-3′,5′-cyclic monophosphate (6-Bnz-cAMP), an analogue of cAMP, is useful as an agent for improved surgical bone repair and regeneration.

### BACKGROUND

Bone repair and regeneration using scaffolds of biodegradable biomaterials together with cells and/or growth factors is an active area of research. Initial adhesion of cells to the scaffold followed by subsequent proliferation, and differentiation of cells into bone, usually with concomitant scaffold degeneration is essential to successful bone repair or regeneration. Synthetic polymeric scaffolds such as polylactide (PLA), polyglycolide (PGA) and their co-polymers poly(lactide-co-glycolide) (PLAGA) have been used successfully as bone repair and regeneration scaffolds. Due to its biodegradable nature, PLAGA has been fabricated into microparticles for loading hydrophilic drugs to be released in a controlled manner. PLAGA microparticles have also been sintered by and molded into structures for bone repair and regeneration.

Bone morphogenetic proteins (BMPs) are a class of osteoinductive growth factors formulated with polymeric biomaterials for bone repair and regeneration. Bone morphogenetic protein 2 (BMP-2) supports the attachment, growth and differentiation of preosteoblasts into osteoblast-like cells under controlled culturing conditions. After sufficient bone tissue has formed ex vivo, the cultured cells can then be implanted into a patient. However, there are several disadvantages to using BMP-2 protein. Like other proteins, BMP-2 can be subjected to enzymatic degradation in vivo. Moreover, purified recombinant BMP-2 protein is expensive (approximately US$ 40/μg). In addition, BMP-2 protein may cause an unwanted immune response or other unwanted side effects in the host. Thus there exists a need for compounds that promote cell attachment, growth, and differentiation into bone without the expense and unwanted side effects associated with BMP-2 protein.

The following publications describe the understanding of the roles of cAMP, PKA and bone morphogenetic proteins (BMPs) in the differentiation of cell types related to bone formation: Siddappa R., et al., “cAMP/PKA pathway activation in human mesenchymal stem cells in vitro results in robust bone formation in vivo” PNAS USA (2008) 105(20): 7281-7286, Ghayor C., et al., “cAMP enhances BMP2-signaling through PKA and MKP1-dependent mechanisms.” Biochem. Biophys. Res. Comm. (2009) 381(2): 247-252; and Rangarajan S, Enserink J. M., et al., “Cyclic AMP induces integrin-mediated cell adhesion through Epac and Rap1 upon stimulation of the beta 2-adrenergic receptor.” J. Cell Biol. (2003) 160(4): 487-493.

Cyclic adenosine monophosphate (cAMP), a small signaling molecule, is found ubiquitously in mammalian cells and acts as a common second messenger controlling diverse cellular processes such as cell adhesion, cell cycle control, and cell differentiation. Protein kinase A (PKA) is one of the sensors for cAMP, resulting in the phosphorylation of a large variety of downstream proteins and, in turn, regulating numerous cellular events including cell-substrate adhesion, proliferation, and differentiation which are the elementary events for bone regeneration and repair.

N⁶,2′-O-dibutyryladenosine-3′,5′-cyclic monophosphate (db-cAMP), a target non-specific cAMP analogue, enhances in vitro osteogenesis and in vivo bone formation via the Protein Kinase A (PKA) pathway. Because db-cAMP can activate all cAMP receptors non-specifically in vivo, activation of unwanted cAMP signaling pathways by db-cAMP may cause a variety of side effects in the host. Moreover, db-cAMP is known to inhibit cell proliferation (non osteo-proliferative), an action detrimental to successful bone tissue engineering.

### SUMMARY

N⁶-Benzoyladenosine-3′,5′-cyclic monophosphate (6-Bnz-cAMP) is a target specific analogue of natural cAMP that has been shown to bind to and to activate the Protein Kinase A (PKA) signaling cascade exclusively. The present disclosure establishes that 6-Bnz-cAMP can serve as a bioactive factor, promoting cell attachment, proliferation, and differentiation of pre-osteoblasts. The disclosure provides the use of 6-Bnz-cAMP to produce osteo-proliferative and osteo-inductive (bone formation) effects in pre-osteoblast cells, including MC3T3E1 cells.

N⁶-Benzoyladenosine-3′,5′-cyclic monophosphate (6-Bnz-cAMP) is a small molecule cAMP analog having the following structure:

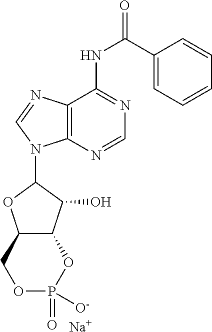

6-Bnz-cAMP is shown here as the sodium salt, which is a common commercially available form. However methods of using other pharmaceutically acceptable 6-Bnz-cAMP salts, hydrates, and solvates, and the 6-Bnz-cAMP free base to promote bone repair and regeneration are within the scope of this disclosure.

In another aspect a bone tissue engineering scaffold, such as a PLAGA microsphere-based formulation, for delivering 6-Bnz-cAMP in vivo is provided by the disclosure.

The disclosure provides method for inducing bone formation in a patient in need thereof comprising administering an effective amount of benzoyladenosine-3′,5′-cyclic monophosphate (6-Bnz-cAMP) to the patient. The disclosure also provides a method of bone repair or replacement comprising: contacting pre-osteoblast cells on PLAGA scaffolds with an effective concentration of N⁶-Benzoyladenosine-3′,5′-cyclic monophosphate (6-Bnz-cAMP).

This disclosure also provides a system for bone tissue engineering, the system comprising: a polymer-based scaffold and 6-Bnz-cAMP.

The disclosure also discloses a method of preparing osteoblasts or osteoblast-like cells for use in bone tissue engineering comprising contacting pre-osteoblast cells on PLAGA scaffolds with an effective concentration of N⁶-Benzoyladenosine-3′,5′-cyclic monophosphate (6-Bnz-cAMP); promoting initial cell adhesion and cell proliferation of such cells, and thereby inducing differentiation of cells to osteoblasts or osteoblast-like cells on PLAGA scaffolds. Osteoblast-like cells have the behavior similar to primary osteoblasts. They exhibit osteoblast differentiation and mineralization after growth in certain conditions. The disclosure also provides a method of using the osteoblasts or osteoblast-like prepared on PLAGA scaffolds for bone grafting, surgical bone repair, a bone replacement, bone regeneration, or a combination thereof.

### BRIEF DESCRIPTION OF THE DRAWINGS

FIG. 1. Schematic representation of the signaling cascade activated by cell permeable cAMP analogue, 6-Bnz-cAMP. Protein Kinase A (PKA) is the exclusive receptor for 6-Bnz-cAMP. Unlike cAMP, 6-Bnz-cAMP does not activate the Epac-Rap1 pathway. P-CREB denotes phosphorylated cAMP response element-binding protein; β-MAPK denotes phosphorylated mitogen-activated protein kinase; and Runx2 is a bone-specific transcription factor.

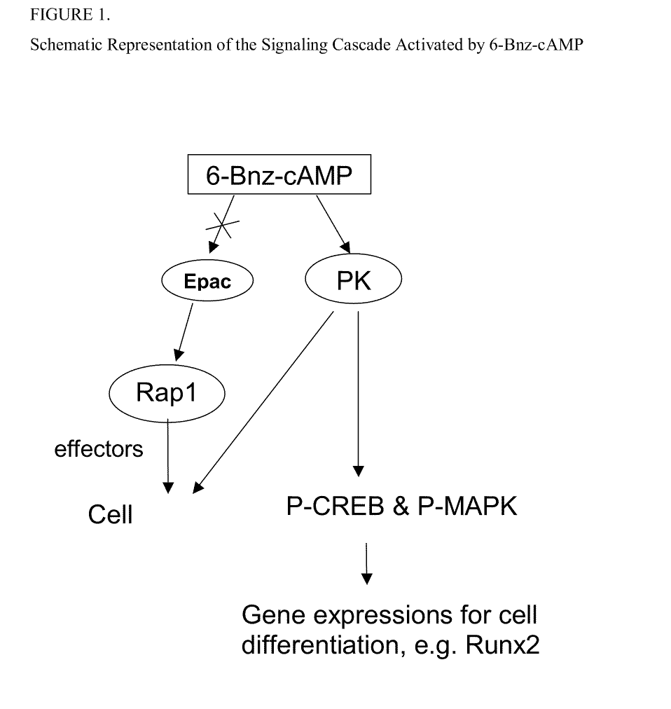

FIG. 2. Effects of 6-Bnz-cAMP on cell adhesion in MC3T3E1 on PLAGA matrices. Cells were treated with 100 μM 6-Bnz-cAMP or untreated. Cells were allowed to adhere for at least 1 h, and nonadherent cells were removed by washing with phosphate buffer saline (PBS). The adhered cells were stained with crystal violet solution for 15 minutes at room temperature and the absorbed stain dye was then solubilized in 1% SDS solution. The amount of dye taken up by the cells was measured in a spectrophotometer. The plots shown are representative of two independent experiments, each done in triplicate (n=3). Error bars represent SD.

FIG. 3. 6-Bnz-cAMP promotes cell growth in the earliest day of cell culture (Day 1) and maintains the cell proliferation rate at Day 7 and Day 14. Error bars represent SD.

FIG. 4. 6-Bnz-cAMP induces the expression of Runx2 in the absence of osteogenic medium Samples were collected at day 21. It is thought that Runx2 serves as a gatekeeper to promote osteogenesis, thus it is one of the most important marker for osteogenesis. In the control panel, consistent with the reported data in literature, Runx2 was detected in the presence of osteogenic medium but remain undetectable level without the osteogenic medium. Note that the addition of osteogenic medium together with 6-Bnz-cAMP further enhanced the expression of Runx2 over that observed with 6-Bnz-cAMP alone. The blot of tubulin serves as loading control for each sample loading.

FIG. 5. 6-Bnz-cAMP increases ALP activities at day 7 and 14. ALP activities of MC3T3-E1 cells were induced by 6-Bnz-cAMP at day 7 and further increased at day 14. To facilitate the comparison of different experimental settings, the ALP activity of the cells cultured in the regular growth medium at Day 7 was set to one relative unit.

FIG. 6. 6-Bnz-cAMP promotes osteoblastic mineralization by day 21. After 20 days of culture of osteoblast-like MC3T3-E1 cells in mineralization medium with or without 6-Bnz-cAMP, quantitative calcium levels were determined. Calcium levels were significantly elevated in osteoblasts cultured in mineralization medium. Osteoblast-like MC3T3-E1 cells cultured in mineralization medium supplemented with 6-Bnz-cAMP further enhanced calcium deposition when compared to the cells cultured in the mineralization medium alone. To facilitate the comparison of different experimental settings, the level of calcium measurement of the “Control” cells cultured in the regular growth medium was set to one relative unit.

FIG. 7. MC3T3-E1 cell viability of cells treated with 6-Bnz-cAMP compared to untreated cells. Cell viability was measured by trypan blue viability stain. No significant differences were found when comparing between the 6-Bnz-cAMP treated and the untreated control cells at each time point, indicating that the viability of MC3T3-E1 cells is not affected by the 6-Bnz-cAMP treatment.

FIG. 8. Effect of 6-Bnz-cAMP on the proliferation of rat mesenchymal stem cells seeded on PLAGA scaffolds. Cell proliferation was assessed by PicoGreen dsDNA assay. The assay demonstrated that 6-Bnz-cAMP treated and untreated groups supported cell proliferation from day 7 to day 21. No significant differences were found when comparing between the 6-Bnz-cAMP treated and the untreated control cells at each time point, indicating that the viability of cells is not affected by the 6-Bnz-cAMP treatment.

FIG. 9. Quantitative calcium levels determined in untreated (control) rat mesenchymal stem cells and cells treated with BMP-2 or 6-Bnz-cAMP. Keys: D-BMP-2 peptide; E-6-Bnz-cAMP; F-Control. Rat mesenchymal stem cells cultured medium supplemented with 6-Bnz-cAMP further enhanced calcium deposition when compared to the control untreated cells and cells supplemented with BMP-2 peptide.

FIG. 10. ALP activity in untreated (control) rat mesenchymal stem cells and cells treated with BMP-2 or 6-Bnz-cAMP. A-BMP-2 peptide; E-6-Bnz-cAMP; F-Control. 6-Bnz-cAMP increases ALP activity. ALP activities of mesenchymal stem cells were induced by 6-Bnz-cAMP at day 7.

### DETAILED DESCRIPTION TERMINOLOGY

The use of the terms “a” and “an” and “the” and the like in the context of describing the invention (especially in the context of the claims included herein) are to be construed to cover both the singular and the plural, unless otherwise indicated herein or clearly contradicted by context. The terms “comprising”, “having”, “including”, and “containing” are to be construed as open-ended terms (i.e., meaning “including, but not limited to”) unless otherwise noted. Recitation of ranges of values herein are merely intended to serve as a convenient method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and is not intended to pose a limitation on the scope of the invention.

6-Bnz-cAMP, 6-Bnz-cAMP and 6 Bnz-cAMP all refer to N⁶-Benzoyladenosine-3′,5′-cyclic monophosphate. The 6-Bnz-cAMP may be used as a free base or pharmaceutically acceptable salt. In certain embodiments the sodium salt of 6-Bnz-cAMP is used.

“Pharmaceutically acceptable salts” includes derivatives of the disclosed compounds, wherein the parent compound is modified by making non-toxic acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues such as carboxylic acids; and the like, and combinations comprising one or more of the foregoing salts. The pharmaceutically acceptable salts include non-toxic salts and the quaternary ammonium salts of the parent compound formed, for example, from non-toxic inorganic or organic acids. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts.

Pharmaceutically acceptable organic salts include salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like, and combinations comprising one or more of the foregoing salts.

“Particles” may be of any shape, while “microspheres” are spherical.

No language in the specification should be construed as indicating that any non-claimed element is essential to the practice of the invention. The terms and expressions that have been employed herein are used as terms of description and not of limitation, and there is no intention in the use of such terms and expressions of excluding any equivalents of the features shown and described herein or any portions thereof. It is recognized that various modifications are possible within the scope of the invention claimed.

### Compositions

It is an object of the disclosure to provide improved compositions and methods to enhance the differentiation of mesenchymal stem cells into osteoblasts. The inventors have discovered certain cAMP analogues, including 6-Bnz-cAMP are useful for inducing mesenchymal stem cells and other pre-osteoblast cells to exhibit the osteoblast phenotype. Thus, as demonstrated herein, 6-Bnz-cAMP can be used to induce MC3T3E1 cells to exhibit the osteoblastic phenotype and support attachment and growth of MC3T3E1 cells.

Microsphere-based scaffolds containing a cAMP analogue, such at 6-Bnz-cAMP, useful for bone grafting and supporting bone regeneration are provided by this disclosure. The utility of the bone regeneration scaffolds provided herein is demonstrated in part by drug release kinetic studies, and by the in vivo performance of scaffolds.

### Methods of Treatment

This disclosure provides a method of using a cell-permeable small molecule cAMP analogue capable of activating protein kinase A (PKA) and thereby promoting attachment, cell adhesion, proliferation and differentiation of pre-osteoblasts and mesenchymal stem cells in vivo and also in vitro. In certain embodiments the cAMP analogue is N⁶-Benzoyladenosine-3′,5′-cyclic monophosphate (6-Bnz-cAMP).

The cAMP analogues, particularly 6-Bnz-cAMP, disclosed herein for bone regeneration and repair may be applied to bone repair scaffolds to enhance osteoblast formation.

A method for inducing bone formation, typically for bone repair or regeneration, in a patient in need thereof comprising administering an effective amount of benzoyladenosine-3′,5′-cyclic monophosphate (6-Bnz-cAMP) or a pharmaceutically acceptable salt thereof to the patient is provided by this disclosure. In this context “administering” means application of 6-Bnz-cAMP, a salt or close analogue thereof, directly to the bone. Typically routes of administration include implanting a polymeric scaffold comprising 6-Bnz-cAMP at a site in the bone where bone repair or regereneration is needed.

In addition to 6-Bnz-cAMP, close analogues of 6-Bnz-cAMP may be used for any of the methods provided by this disclosure. Suitable 6-Bnz-cAMP analogues include those in which the 6-benzyl group has been conservatively substituted, for example with a halogen, hydroxyl, C₁-C₂alkyl, or C₁-C₂alkoxy. Close analogues particularly include those in which the benzyl group or adenosie is substituted with a methyl. Another class of 6-Bnz-cAMP analogues in which the 6-benzyl is optionally substituted with a conservative substituent, e.g. halogen, hydroxyl, C₁-C₂alkyl, or C₁-C₂alkoxy, and the adenosine is also substituted with a conservative substituent, such as C₁-C₂alkyl or C₁-C₂alkoxy is included in this disclosure.

Methods of using cAMP analogues include 6-Bnz-cAMP, to stimulate differentiation of mesenchymal stem cells and pre-osteoblast cells into osteoblast-like cells are provided by this disclosure. cAMP analogues provided by this disclosure stimulate osteoblast differentiation and mineralization from mesenchymal stem cells to effect bone regeneration and repair. The use of a small molecule to induce bone regeneration and repair has a number of advantages over present methods, which use growth factors such as bone morphogenetic proteins (BMP). BMP shortcomings include protein instability, including thermal instability, high cost, immunogenicity, and need for supraphysiological dosage. cAMP analogues, such as 6-Bnz-cAMP, do not have these limitations. In contrast 6-Bnz-cAMP is a thermally stable molecule with a higher melting point than BMP-2 (˜250° C.). 6-Bnz-cAMP also has a long half-life because it was specially designed to resist enzymatic degradation in vivo; 6-Bnz-cAMP is resistant to hydrolysis by phophodiesterase. Moreover, 6-Bnz-cAMP has the advantage of being soluble in water and a variety of pharmaceutically useful buffers and solvents.

Compared to the non-specific cAMP analogue, db-cAMP, 6-Bnz-cAMP selectively activates the Protein Kinase A (PKA) pathway, thus minimizing the unwanted side effects that can occur with non-specific activation of multiple cAMP receptors by non-specific cAMP analogues. 6-Bnz-cAMP supports multiple aspects of cellular development from initial cell adhesion and cell growth, to cell differentiation. These features of 6-Bnz are in sharp contrast to db-cAMP, which is known to inhibit cell growth and 8-Br-cAMP which inhibits osteoblast proliferation. Further, compared to BMPs, 6-Bnz-cAMP is thermally more stable as it has a higher melting point. Taken together, these intrinsic properties make 6-Bnz-cAMP an extremely attractive candidate for therapeutic purposes as part of a matrix system for bone regenerative engineering.

The disclosure provides a method for inducing bone formation in a patient in need thereof comprising administering an effective amount of benzoyladenosine-3′,5′-cyclic monophosphate (6-Bnz-cAMP) to the patient.

The disclosure also includes a method of tissue engineering comprising:

contacting pre-osteoblast cells on a polymer scaffold, such as a PLAGA scaffold or polysaccharide scaffold, with an effective concentration of N⁶-Benzoyladenosine-3′,5′-cyclic monophosphate (6-Bnz-cAMP). The method bone tissue engineering is further a method increasing proliferation of osteoblasts, a method of proliferation of osteoblast-like cells, a method of bone grafting, a method of surgical bone repair, a method of bone replacement, a method of bone regeneration, or a combination thereof. In certain embodiments the concentration of 6-Bnz-cAMP is a concentration sufficient to activate the Protein Kinase A pathway, for example a concentration of about 100 μM or about 50 μM to about 200 μM. The method may further comprise contacting the pre-osteoblast cells, at the same time they are in contact with 6-Bnz-cAMP, with Bone Morphogenetic Protein 2 (BMP-2) at a concentration that is about 10% of the concentration of BMP-2 needed for optimal osteoinduction by BMP-2 in the absence of 6-Bnz-cAMP. In certain embodiments of this disclosure the pre-osteoblast cells are mesenchymal stem cells.

The disclosure also provides a method of preparing osteoblasts or osteoblast-like cells for use in bone tissue engineering comprising: contacting pre-osteoblast cells on scaffolds, including PLAGA scaffolds, with an effective concentration of N⁶-Benzoyladenosine-3′,5′-cyclic monophosphate (6-Bnz-cAMP); promoting initial cell adhesion and cell proliferation of such cells, and thereby inducing differentiation of cells to osteoblasts or osteoblast-like cells on scaffolds. The pre-osteoblast cells may be mesenchymal stem cells.

The disclosure also provides a method of using the osteoblasts or osteoblast-like cells prepared by any method disclosed herein or known to those of skill in the art, in vivo in a method of treatment chosen from bone grafting, surgical bone repair, bone replacement, bone regeneration, or a combination thereof.

### Scaffolds

A wide range of synthetic and natural polymers have already been adopted for 3-D scaffold fabrication. Synthetic biodegradable polymers such as poly(esters), poly(anhydrides), poly(anhydride-co-imides) and poly(phosphazene) derivatives, have been used to fabricate scaffolds for bone repair. Synthetic materials useful as bone regeneration scaffolds fabrication due to their programmable degradation characteristics are known in the art. See, for example, Laurencin, et al., in Annual Review of Biomedical Engineering, Yarmush (ed.) Annual Reviews Inc., Palo Alto, (1999) 1: 19-46). The α-hydroxyesters poly(lactic acid) (PLA), poly(glycolic acid) (PGA) and their copolymer PLAGA are approved by Food and Drug Administration (FDA) for certain biomedical applications (Athanasiou, et al., Arthroscopy, (1998) 14: 726-737). Suitable polymer based scaffolds also include polysaccharide-based scaffold such as those disclosed in US2010-0249931, which is hereby incorporated by reference in its entirety, and particularly at paragraphs [0033] to [0066] and Examples 1-3, for its teachings regarding polysaccharide scaffolds.

Further methods and compositions for preparing scaffolds comprising a biodegradable, biocompatible polymer and specific information regarding PLAGA as an example of such a polymer are described in U.S. Pat. No. 5,866,155 to Laurencin and Borden, which is hereby incorporated by reference for its teachings regarding biodegrable and biocompatible polymer matrices for bone regeneration. In one embodiment, a bone grafting system or bone repair system comprising PLAGA microsphere matrices and 6-Bnz-cAMP, is provided. The biological performance of the 6-Bnz-cAMP loaded, microsphere-based scaffold is examined in vivo using a rabbit ulnar critical size defect model. In this embodiment the bioactivity of a scaffold system that comprises PLAGA microspheres is improved by the incorporation of the small molecule 6-Bnz-cAMP in the system. In a further embodiment, use of a small molecule/polymeric matrix system results in enhanced bone regeneration and bone repair in vivo. The process of bone formation in such implants is characterized by a variety of techniques including radiography, microcomputed tomography, and histology.

The disclosure provides a system for bone tissue engineering, the system comprising:

a polymer-based scaffold and 6-Bnz-cAMP. It is preferred that the polymer is a degradable and biocompatible polymer, such as poly(lactic-co-glycolide) (PLAGA) or a polysaccharide based polymer. In certain embodiments the system is one in which the 6-Bnz-cAMP is present at a concentration sufficient to activate the Protein Kinase A (PKA) pathway in pre-osteoblast cells. In certain embodiments the polymer-based scaffold comprises microspheres, such as PLAGA microspheres or sintered polysaccharide microspheres. The system may also include cells such mesenchymal stem cells having osteo-proliferative and osteo-inductive responses to 6-Bnz-cAMP. The bone tissue engineering system may also include Bone Morphogenetic Protein 2 (BMP-2) at a concentration that is about 10% of the concentration of BMP-2 needed for optimal osteoinduction by BMP-2 in the absence of 6-Bnz-cAMP.

This disclosure also provides a 6-Bnz-cAMP loaded microsphere-based scaffold for bone repair and regeneration. In certain embodiments the 6-Bnz-cAMP-loaded microsphere-based scaffold is a 6-Bnz-cAMP sustained release system or controlled-release delivery system. Methods for incorporating biomolecules, including small molecule growth factors such as 6-Bnz-cAMP, into biodegradable polymer delivery systems, are well known in the art. See for example John, P. M., et al. (1979) J. Pharm. Sci., 68(4): 475-481., Lin, S. Y., et al., (1985) Biomater Med. Devices Artif. Organs, 13(3-4): 187-201, Cohen, S., et al., (1994) Int. J. Technol., Assess Health Care, 10(1): 121-130., Alonso, M. J., et al., (1994) 12(4): 299-306., Boisdron-Celle, M., et al., (1995) 47(2): 108-114., Birnbaum, D. T., et al., J. Control Release (2000) 65(3): 375-387., Cryer, S. A, et al., (2009) J. Agric. Food Chem., 57(12): 5443-5451. Certain polymer bone repair and replacement scaffolds are created using stringent organic solvents, cross-linking reagents, and/or high temperature. Such harsh conditions make incorporating biomolecules into the scaffold without denaturation or deactivation problematic. As discussed above the small molecule 6-Bnz-cAMP is inherently more stable than the protein growth factors presently used to stimulate bone repair and regeneration and does not suffer the same limitations for incorporation into a sustained release system.

### EXAMPLES

### Materials

N⁶-benzoyladenosine-3′,5′-cyclic monophosphate (6-Bnz-cAMP) was purchased from Biolog Life Science Institute (Bremen, Germany); anti-Runx2 antibody was purchased from Invitrogen (Camarillo, Calif.); anti-phosho-CREB (1B6), anti-phospho-Erk1/2, anti-Erks antibodies were purchased from Cell Signaling Technology Inc (Temecula, Calif.); anti-β-tubulin (clone AA2) antibody was purchased from Millipore (Billerica, Mass.); anti-osteopontin antibody (AKm2A1), anti-osteocalcin (FL-95) antibody and Protein A/G agarose were purchased from Santa Cruz Biotechnology, Inc (Santa Cruz, Calif.). Horseradish peroxidase (HRP)-labeled affinity purified antibodies to mouse IgG and rabbit IgG were purchased from KPL (Gaithersburg, Md.). Chemiluminescent substrate for detection of HRP and X-Ray films were purchased from Thermo Scientific (Rockford, Ill.).

### Example 1

### Adhesion, Proliferation, Viability, and Differentiation of MC3T3E1 Cells Treated with 6-Bnz-cAMP

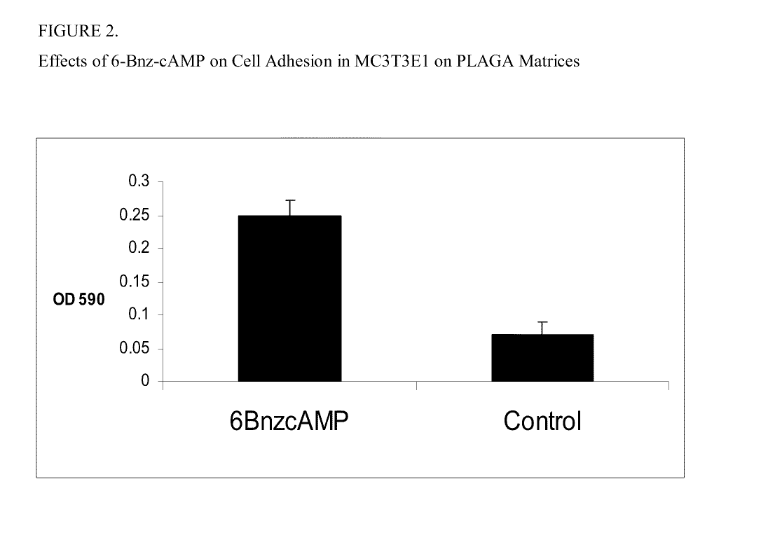

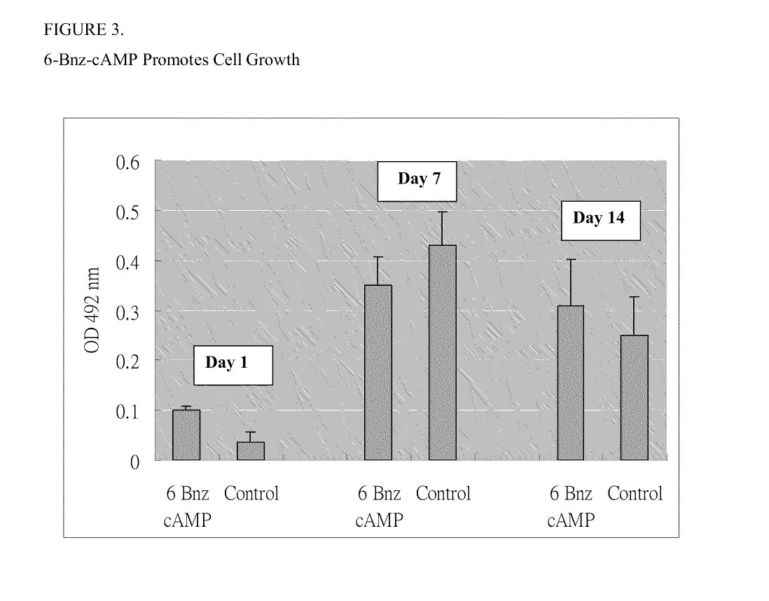

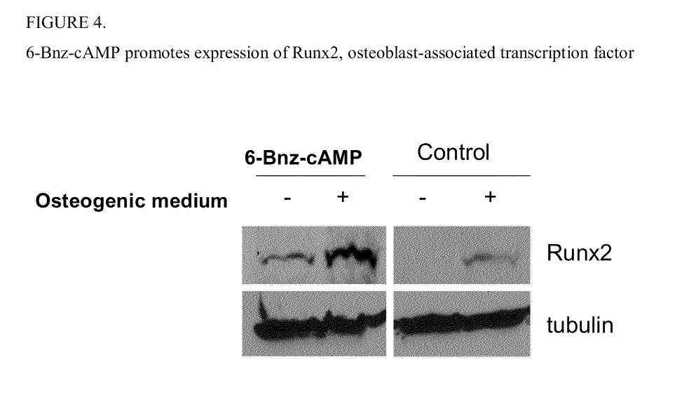

The feasibility of using 6-Bnz-cAMP in bone tissue engineering was demonstrated under controlled cell culture conditions. For these experiments, the polymer component of the scaffold, poly(lactic-co-glycolide) (PLAGA) was selected because of its documented degradability and biocompatibility. MC3T3E1 cells were chosen as a study model system because this cell type is fast-growing and displays classic osteoblast markers when the cells differentiate. 6-Bnz-cAMP was added to the culture medium to a final concentration of 100 μM for experiments shown in FIGS. 2 through 4,. In vitro, 6-Bnz-cAMP was found to support the attachment (FIG. 2), growth (FIG. 3), and differentiation (FIG. 4) of MC3T3E1 cells into osteoblast-like cells.

More specifically, thin film discs of poly(lactic-co-glycolide) (PLAGA) were fabricated using a traditional solvent-casting method. In this process, the polymer was first dissolved in methylene chloride, then poured into a Teflon-coated dish. The dishes were then placed in a −20° C. freezer to allow solvent evaporation. The thin film matrices were subsequently bored into 1.0 cm diameter discs.

MC3T3E1 subtype 4 cell line was purchased from the American Type Cell Collection (ATCC). The cells were grown to confluence, then seeded onto PLAGA discs at a density of 50,000 cells/scaffold. The cells were cultured on the discs in vitro in a 37° C. and 5% CO₂ environment, using MEM alpha cell culture medium +10% Fetal Bovine Serum as a nutrient source. At specific time points, cultured cells were collected for various assays i.e., cell adhesion assays, cell proliferation assays, and cell differentiation studies.

Adhesion: In the cell adhesion study presented in FIG. 2 cells were allowed to adhere for at least 1 h, and nonadherent cells were removed by washing with phosphate buffer saline (PBS). The adhered cells were stained with crystal violet solution for 15 minutes at room temperature and the absorbed stain dye was then solubilized in 1% SDS solution. The amount of dye taken up by the cells was measured in a spectrophotometer. As shown in FIG. 2, 6-Bnz-cAMP enhances the initial cell adhesion of MC3T3E1 cells on PLAGA matrices.

Proliferation: Cell proliferation was tested using a PicoGreen dsDNA assay kit (Molecular Probes, Eugene, Oreg.) The procedure of the PicoGreen dsDNA assay was performed according to the manufacturer's instructions. Briefly, 5×10⁴ cells ml⁻¹ were seeded in a well of a tissue culture plate. 100 μM of 6-Bnz-cAMP was added to the regular growth medium during cell seeding. The regular growth medium and 6-Bnz-cAMP (100 μM) were replaced every 3 to 4 days. 6-Bnz-cAMP was found to promote cell growth as soon as the first day of culturing (FIG. 3) and the proliferation rate was maintained at Days 7, 14, and 21.

Viability: Cell viability is determined using a trypan blue viability assay. For trypan blue viability assay method, at day 7, 14, and 21, cells were collected by trypsinization and stained with trypan blue dye (MP Biochemicals LLC, Solon, Ohio). The cells were then numbered in a hemacytometer (Hausser Scientific, Horsham, Pa.) under a light microscopy (Olympus Corporation, Tokyo, Japan). Cell stained blue was scored a dead cell, while unstained cell was scored a live cell. The results are presented in % of viability.

Differentiation: Osteogenic protein markers were analyzed SDS-PAGE and Western Blotting. Protein samples were resolved by SDS-PAGE on precast 12.5% polyvinylidene-Tris gels (Bio-Rad, Hercules, Calif.) and transferred electrophoretically onto polyvinylidene difluoride membranes according to the manufacturer's instructions (Bio-Rad, Hercules, Calif.). Membranes were blocked with skim milk and then probed with respective antibodies. Protein bands on blots were visualized by an enhanced chemiluminescent detection kit.

### Example 2

### Matrix Mineralization of MC3T3-E1 Cells Treated with 6-Bnz-cAMP

MC3T3-E1 cells were grown in mineralization media (The minimal media described in Example 1 with, 1% of antibiotic (100 U/ml penicillin G and 100 mg/ml streptomycin), 3 mM β-glycerolphosphate, and 10 μg/ml ascorbic acid). 6-Bnz-cAMP was added to the culture medium to a final concentration of 100 μM for experiments; mineralization media only was used for positive controls. For the qualitative determination of calcified matrix mineralization, at day 21, media were removed and cells were rinsed with calcium-free PBS for 3 times. The precipitated calcium of mineralized matrix was dissolved overnight in 0.6N hydrochloric acid at 4° C. The extracted calcium was then measured with a calcium assay kit (Calcium Liquicolor, Stanbio Laboratory, Boerne, Tex.) according to the manufacturer's instructions. The absorbance values were normalized to cellular DNA.

### Example 3

### In Vitro Efficacy of 6-Bnz-cAMP to Induce Osteogenic Marker Protein Expression in MC3T3-E1 Cells

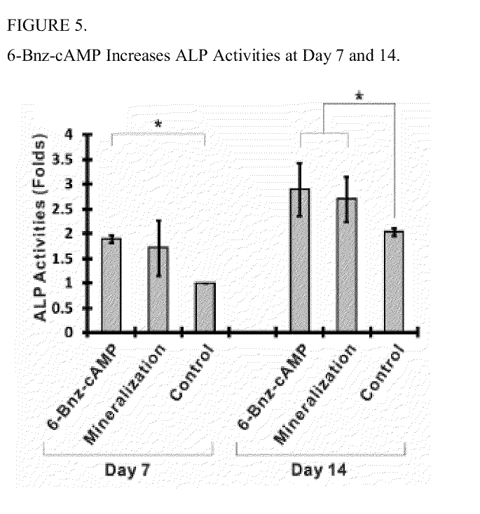

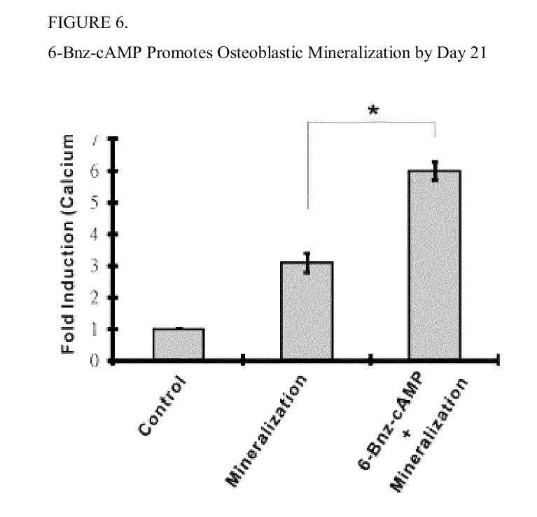

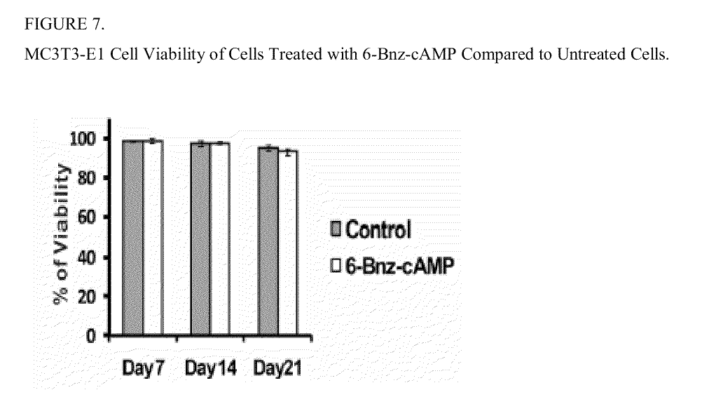

MC3T3-E1 cells were grown in mineralization media. 6-Bnz-cAMP was added to the culture medium to a final concentration of 100 μM for experiments; mineralization media only was used for positive controls. Osteoblast-associated markers were quantitated at days 7 and 14. Markers include increased alkaline phosphatas (ALP) activity (FIG. 5) and calcium precipitation in MC3T3-E1 cells (FIG. 6). 6-Bnz-cAMP toxicity was assessed at days 7, 14, and 21 in cells treated with 100 micromolar 6-Bnz-cAMP compared to untreated cells. Trypan blue staining was used to visualize cells (FIG. 7).

ALP activities were measured using ALP assay kit according to the manufacturer's instructions (Bio-Rad, Hercules, Calif.). Briefly, at day 7 and 14, cells were collected and washed 4 times with PBS prior to the ALP activity assay. Cells were then lysed with 0.1% TX-100. Cell lysates were collected and mixed with the ALP substrate solution for 1 h at 37° C. incubation. The colorimetric change due to the presence of ALP activity was measured by a spectrophotometric plate reader at 405 nm. The absorbance values were then normalized to cellular DNA.

MC3T3E1 cells expressed classic markers for the osteoblastic phenotype in the presence of 6-Bnz-cAMP in the absence of osteogenic medium in samples collected at day 21. The level of Runx2, a bone-specific transcription factor which increases bone formation through the stimulation of bone-marker gene transcription in osteoblasts, was detected by immunoblotting (FIG. 4). In the control panel, Runx2 was detected in the presence of osteogenic medium but remain undetectable level without the osteogenic medium. The addition of osteogenic medium together with 6-Bnz-cAMP further enhanced the expression of Runx2 over that observed with 6-Bnz-cAMP alone. The blot of tubulin serves as loading control for each sample loading.

Extracellular proteins osteocalcin and osteopontin are also determined as a measure of cell differentiaion. The cell culture media after 18 days incubation are collected in microcentrifuge tubes. The extracellullar protein osteocalcin and osteopontin in the culture media was individually isolated by incubating with anti-osteocalcin antibody and anti-osteopontin antibody, respectively. Antibody-bound proteins were then precipitated by incubating with Protein A/G agarose for another 2 h at 4° C. Immune complexes were washed 4 times with PBS to eliminate non-specific bindings. The resulting pellets were resuspended in Laemmli SDS sample buffer (Bio-Rad, Hercules, Calif.) for polyacrylamide gel electrophoresis and Western Blot analysis.

### Example 4

### Osteoinductive Potential of 6-Bnz-cAMP in Rabbit Mesenchymal Stem Cells and Comparison with BMP-2

It has been observed that the small molecule 6-Bnz-cAMP can enhance initial cell adhesion and proliferation. Further, this compound triggers the differentiation of mesenchymal stem cells into osteoblasts. Osteoinductive ability of 6-Bnz-cAMP is shown by measuring the in vitro cellular response of rabbit bone marrow-derived, mesenchymal stem cells. Recombinant bone morphogenetic protein 2 (BMP-2) is employed as a positive control for a direct comparison of the osteoinductive effects of 6-Bnz-cAMP and BMP-2.

Cell isolation: Rabbit bone marrow MSCs are isolated from young male New Zealand white rabbits (average weight: 1.6 kg). Bone marrow cavity contents are aseptically removed and harvested in Dulbecco's Modified Eagle's Medium (DMEM) and 50 μg/ml gentamicin. Marrow is passed through 16 and 20 gauge needles and re-suspended in two medium conditions. Cells are placed in DMEM supplemented with 10% fetal bovine serum, 2.5 mM L-glutamine, and 50 μg/ml gentamicin. Cells are placed in flasks in 20 ml of media and cultured in a humidified 37° C./5% CO₂ incubator. MSCs are selected based on their ability to adhere to the flask while non-adherent hematopoietic cells are removed upon re-feeding after 3 days. The culture medium is replaced 3 times a week.

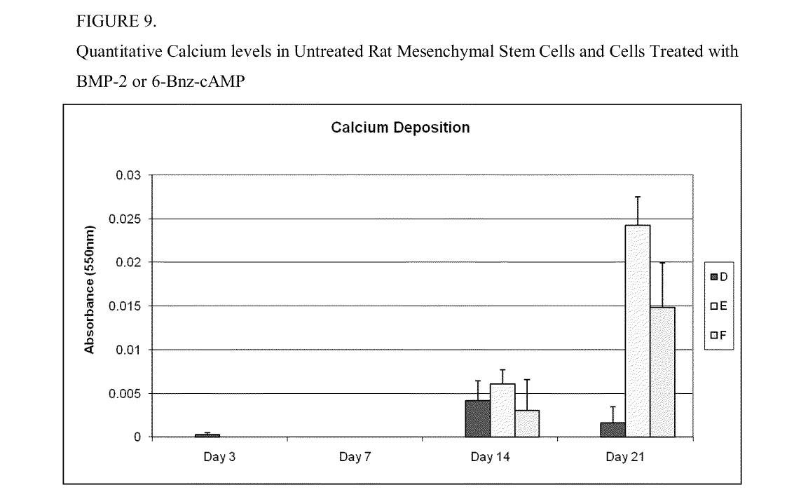

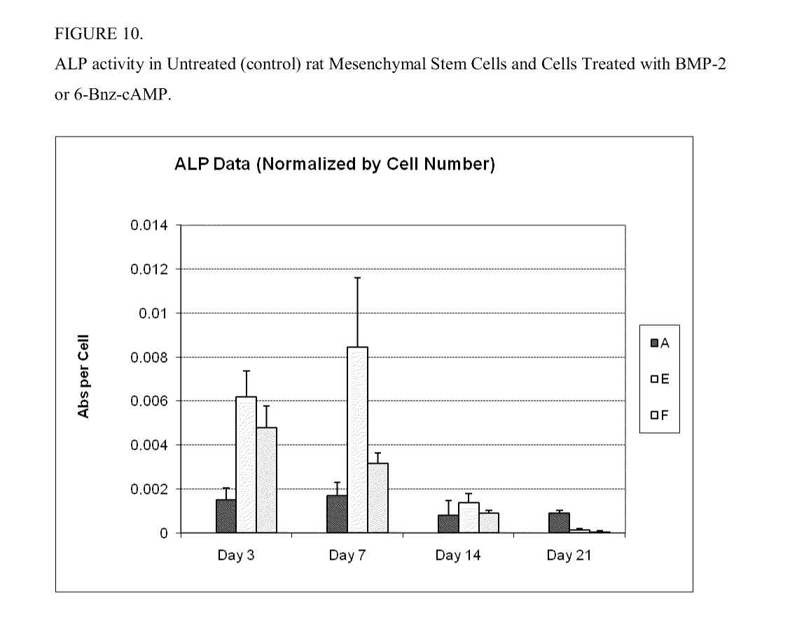

Cell adhesion, proliferation, and differentiation studies: Rabbit MSCs are plated onto PLAGA matrices at a plating density of 1×10⁴ cells/cm² and cultured in standard basal medium. Four experimental groups are tested: (1) untreated cells, (2) 6-Bnz-cAMP treated cells, (3) BMP-2 treated cells, and (4) 6-Bnz-cAMP and BMP-2 treated cells. We use different concentrations of 6-Bnz-cAMP (5 μM, 10 μM, 20 μM, 50 μM, and 100 μM) in order to search for the lowest possible effective concentration to minimize off-target effects of 6-Bnz-cAMP. For BMP-2, we use 50 ng/ml, a concentration known to enhance differentiation in vitro. Initial cell adhesion is determined at 1 h, 3 h, 12 h, and 24 h. Cell proliferation and cell differentiation are determined at 1, 7, 14, and 21 days. Calcium deposition and ALP activity is also measured at days 3, 7, 14, and 21 in untreated cells compared to cells treated with BMP-2 or 6-Bnz-cAMP (FIGS. 9 and 10). The culture medium is replaced every 3 days. The bio-factors (6-Bnz-cAMP and BMP-2) are administrated continuously. At the predetermined times, the scaffolds are removed from the culture and gently washed with PBS 4 times to remove unattached cells. For the cell adhesion assay, attached cells are quantified by crystal violet stain, a method routinely used to measure the initial cell adhesion. Cell proliferation is measured using a commercially available proliferation assay kit, MTS (Promega). For alkaline phosphatase (ALP) activity, the early markers of osteoblast differentiation are determined with a commercially availia ALP assay kit (Bio-rad). Osteoblast differentiation-related genes are identified in cells cultured on the scaffolds by total RNA isolation and the RT-PCR techniques. At the predetermined times, cells are harvested using trypsin-EDTA solution. Total RNA is isolated using the Quick Prep Total RNA extraction kit (Qiagen). Specific primer pairs for runx2, osterix, osteocalcin, osteopontin, and the housekeeping gene will be designed. PCR amplification will be carried out and the resulting products will be analyzed by DNA gel electrophoresis and sequencing. Protein gel electrophoresis and Western Blot analysis will be carried out to detect osteoblast-associated protein expressions such as runx2, osterix, bone sialoprotein, osteocalcin and osteopontin protein productions.

Mineralization: The same experimental groups and conditions described above except are utilized except that the cells are cultured in osteogenic medium (Lonza) without dexamethasone supplement. Scaffolds are removed from media at days 7, 14, 21, and 28, washed with Ca²⁺ free PBS 4 times, and stained with Alizarin Red (Sigma). Energy dispersive X-ray analysis is used to confirm the presence of calcium and phosphorus in the scaffolds.

### Example 5

### In Vitro Fabrication, Characterization, and Evaluation of 6-Bnz-cAMP Loaded PLAGA Microspheres Scaffolds

In this example the small molecule 6-Bnz-cAMP is combined with PLAGA to form a scaffold delivery system that provides controlled release of 6-Bnz-cAMP from the scaffold system. 6-Bnz-cAMP is loaded into PLAGA microspheres using a double emulsion solvent evaporation technique. The 6-Bnz-cAMP loaded PLAGA microspheres are then sintered into a 3-dimensional scaffold. “Sintering” is the thermal treatment of a powder or compact at a temperature below the melting point of the main constituent, for the purpose of increasing its strength by bonding together of the particles. “Sintered” materials are any materials that have been formed by the process of sintering.

6-Bnz-cAMP loaded PLAGA microspheres are fabricated using a double emulsion solvent evaporation technique. This previously reported technique is useful for loading both water soluble and insoluble therapeutics. (McGinity, J. W. and O'Donnell, P. B., Adv Drug Del. Rev. (1997) 28(1): 25-42., Zhu, K. J., et al., J. Microencapsul., (2001) 18(2); 247-260.) In brief, a weighted quantity of 6-Bnz-cAMP in 200-400 μL of phosphate buffered saline is dispersed in 3 ml of (20%) PLAGA solution by vigorous agitation/sonication to create a drug polymer primary emulsion. This primary emulsion is added in a thin stream to 200 ml of 1% PVA solution to create a secondary emulsion at a constant stirring speed using a magnetic stirrer overnight. Hardened drug loaded microspheres are isolated by vacuum filtration and washed repeatedly with deionized water, air-dried for 2 h and then kept desiccated until further use. Microspheres are segregated using standard sieves. Microspheres in the size range of 600-710 microns are used for fabricating scaffolds. A control PLAGA microsphere based scaffold constructed using the same protocol except that it does not contain 6-Bnz-cAMP is used as a control.

The loading efficiency is determined by dissolving the 6-Bnz-cAMP loaded scaffolds in methylene chloride, drying under a fume hood, and weighing to determine the mass associated with remaining 6-Bnz-cAMP. The external morphology of microspheres is characterized by Scanning Electron Microscopy (SEM). The other basic physical properties of the PLAGA microsphere-based scaffolds are characterized using techniques using compression testing (for measurements of strength and modulus) and matrix degradation studies. These standard techniques and procedures are routinely performed using techniques known in the art.

In vitro 6-Bnz-cAMP release study: The scaffolds are soaked in MEM medium with 10% FBS at 37° C. Equal aliquots of the medium are withdrawn daily and replaced by an equal volume of fresh pre-warmed medium up to 30 days. At day 31, the scaffolds are placed into a lysis buffer (50 mM Tris-HCl, 1% Triton X-100, 0.1% SDS, pH 5.5) and homogenized for 5 min. The homogenate is subjected to centrifugation and the supernatant collected. The amounts of 6-Bnz-cAMP in both collected medium and the supernatant is determined using a cAMP ELISA kit (R&D systems, Inc).

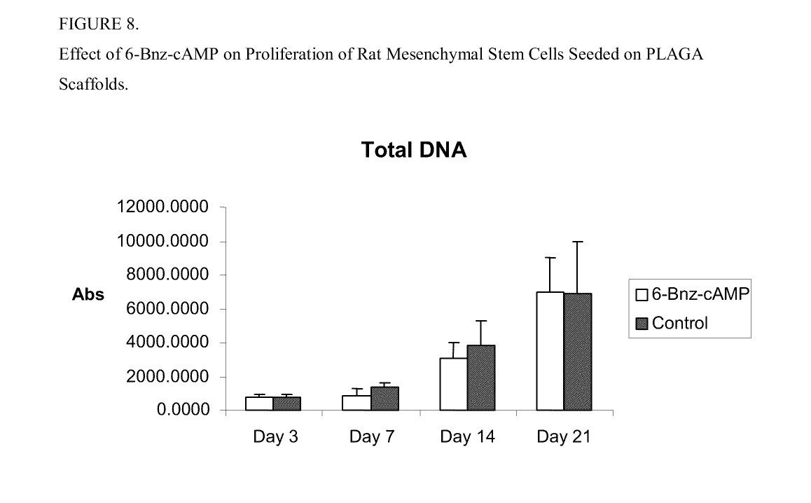

Evaluation of the in vitro cellular behaviors of rabbit MSC on the 6-Bnz-cAMP loaded PLAGA microsphere based scaffold: For in vitro studies, cells are plated onto the 6-Bnz-cAMP loaded and unloaded control scaffolds. Cell adhesion is determined at 1, 3, 6 and 24 hours. Cell proliferation levels are determined at Day 3, 7, and 14 (FIG. 8). Cell differentiation and mineralization are determined at Day 7, 14, 21, and 28. At the pre-determined times, scaffolds will be removed from the medium and unattached cells are removed by washing with PBS 4 times. The in vitro assays are performed on the attached cells according to the procedures described above in Example 3.

### Example 6

### In Vivo Use and Biological Performance of 6-Bnz-cAMP Loaded Microsphere Scaffold

6-Bnz-cAMP is released from microsphere scaffold systems and causes osteogenesis and/or accelerates bone regeneration in vivo. More specifically, bone forming and bone inducing capacity of the 6-Bnz-cAMP loaded PLAGA microsphere scaffold is characterized in a rabbit ulnar critical size defect model. The rabbit ulnar critical size defect model has been extensively used to evaluate bone regeneration capacity of implants. This model is also used to assess the biological performance of 6-Bnz-cAMP loaded microsphere scaffolds in vivo. The following studies demonstrate that 6-Bnz-cAMP released from the microspheres scaffold system can cause osteogenesis and accelerate bone regeneration in vivo.

In vivo bone defect model: For the in vivo studies, the ulnar critical size defect model in adult male New Zealand White Rabbits (3-4 kg) is used. The benefit of choosing ulnar critical size defect model in rabbits is that no post-surgical external fixation is required because the radius acts as a splint for the ulna, allowing the limb to heal with minimal motion. Animal groups and group sizes are as follows:

| Conditions | Number of Animals |
|---|---|
| PLAGA microspheres scaffold | 10 |
| 6-Bnz-cAMP loaded PLAGA microspheres | 10 |
| scaffold | |
| BMP-2 loaded PLAGA microspheres scaffold | 10 |
| 6-Bnz-cAMP/BMP-2 loaded PLAGA | 10 |
| microspheres scaffold | |

Total: 40 × 2 time points = 80 rabbits

Rabbits are allowed to acclimate for 48 hours before surgery after delivery from the vendor. One day prior to surgery, 10 mg/kg Enrofloxacin is given to each rabbit intramuscularly. The rabbits are anesthetized using a mixture of ketamine (50 mg/kg), xylazine (6 mg/kg), and acepromazine (1 mg/kg). The right forelimb of the rabbit is shaved, prepped with betadine and 70% ethanol. The rabbit is covered with a sterile fenestrated drape to expose the surgical site. A longitudinal incision is made above the ulna. Skin and musculature are then be dissected and the mid-diaphysis of ulna is exposed. A segment of the ulna is removed using a reciprocating saw irrigated with 0.9% sodium chloride saline solution. Once the segment of ulna is removed the scaffold is implanted into the defect site. The underlying musculature is then closed using absorbable 3-0 Coated VICRYL* plus antibacterial (polyglactin 910) suture (Ethicon) and the skin will be closed using non-absorbable 3-0 PROLENE* blue monofilament polypropylene suture (Ethicon). For the implants containing BMP-2, the BMP loaded scaffolds are prepared as described in previously (Borden, M., et al., J. Bon Joint Surg. (2004) 86(8): 1200-1208). One μg of recombinant BMP-2 is incorporated onto the surface of the sintered microsphere scaffolds.

The bone healing process is monitored by X-ray analysis at 2, 4, 6, 8, and 10 weeks. Antibiotics and analgesics are administered for 3 and 7 days post-operative, respectively. At the designed time points (6 and 12 weeks), rabbits are euthanized using an overdose of sodium pentobarbital (175 mg/kg). The ulna is isolated for mechanical testing, histological and microcomputed tomography analysis, and to evaluate the potential of 6-Bnz-cAMP off target cell effect.

Mechanical Testing: The degree of healing is evaluated by mechanical testing, using intact bone as a benchmark. For a defect such as we are describing, torsion testing is always employed. Torsional testing to failure is performed by isolating the ulna from the radius, cutting the ulna ˜5 mm proximally and distally to the implant, and securing both ends of the bone in small pots with bone cement. While the cement is curing, the exposed region of bone and implant are kept moist with saline. Once the cement is co-axial with that of the testing device the torsional force is applied at a displacement rate of 50 mm/min with a lever arm of six centimeters, until failure for each specimen. In the event that there is no distinct failure, the point of failure is defined as that point at which the stress has been reduced to 70% of its original value. The torque and angular deformation to failure are obtained from force-angular curves, while the energy absorbed to failure is determined by calculating the area under that curve. Maximum angle of twist is determined as the stress of the torsion vs angular rotation curve at the point of failure.

Microcomputed Tomography (MicroCT): A thresholding analysis is conducted on a single specimen to optimize the equipment test conditions for quantification of bone volume before microCT analysis. For one run of a single scanning cycle on the microCT, the whole rabbit forelimb is soaked in PBS, placed vertically in a 50 ml centrifuge tube which is then placed in the microCT specimen chamber. Radiographs and gross analysis of the specimens are used to locate the defect borders. Scanning of the samples begins just above the upper defect border and will continue for a certain length in order to ensure inclusion of the entire defect area. For the study, 3-D reconstructions of the radius and ulna together are obtained and used to evaluate the new bone formation at the defect site.

Histological Analysis: Limbs are prepared for histology by immersing in 80% ethanol for 24 hours and transferring to fresh 80% ethanol for storage. Samples are washed overnight in running water, dehydrated through alcohols, cleared in xylene, and embedded in methyl methacrylate. These calcified tissues are sectioned at 5-7 microns thickness with a tungsten carbide blade on a Reichert-Jung Ultracut E microtome and mounted onto glass slides. These sections are then be stained with von Kossa to evaluate all the mineralized tissue in the site, and with Goldner's Trichrome to evaluate cellular events, the osteoid, or new un-mineralized bone being deposited at bone forming sites.

The Assessment of Off-target Cell Effects of 6-Bnz-cAMP: We investigate whether the released 6-Bnz-cAMP activates an immune response leading to unintended and off-target effects. Foreign body giant cell (FBGC) formation is a hallmark of the host immune response to implants and they are commonly observed at the tissue/material interface of implant. Histological methods on the retrieved implants are used to identify and quantify multinucleated giant cells of foreign body type. Second, we analyze the tissues surrounding the implants, i.e. muscles and skins, to search for the signs of off-target cell effects of 6-Bnz-cAMP. Specifically, the tissues surrounding the implants are be collected, weighted, homogenized, and analyzed with molecular biology techniques (e.g. Western Blot) to look for specific protein marker(s) for 6-Bnz-cAMP/PKA signaling, particularly pay attention to the pCREB since it is a direct target of cAMP/PKA signaling.

It will be readily apparent to one skilled in the art that varying substitutions and modifications may be made to the invention disclosed herein without departing from the scope and spirit of the invention. For example, the invention comprises any and all cAMP analogues that specifically activate the Protein Kinase A pathway. Therefore, it is intended that the invention not be limited to the particular embodiments and best mode contemplated for carrying out this invention as described herein. Thus, such additional embodiments are within the scope of the present invention and the following claims.

What is claimed is:

1. A method for inducing bone formation in a patient in need thereof comprising administering an effective amount of benzoyladenosine-3′,5′-cyclic monophosphate (6-Bnz-cAMP) or a pharmaceutically acceptable salt thereof to the patient.

2. A system for bone tissue engineering comprising: a polymer-based scaffold and 6-Bnz-cAMP or a pharmaceutically acceptable salt thereof.

3. The system of claim 2, wherein the polymer is degradable and biocompatible.

4. The system of claim 2, wherein the 6-Bnz-cAMP is present at a concentration sufficient to activate the Protein Kinase A (PKA) pathway in pre-osteoblast cells.

5. The system of claim 2, wherein the polymer-based scaffold comprises microspheres.

6. The system of claim 2, wherein the polymer-based scaffold comprises 14. The system of claim 12, wherein the polymer having documented degradability and bio compatibility comprises poly(lactic-co-glycolide) (PLAGA).

7. The system of claim 2, wherein the system further comprises cells and the cells are mesenchymal stem cells having osteo-proliferative and osteo-inductive responses to 6-Bnz-cAMP.

8. The system of claim 2, further comprising Bone Morphogenetic Protein 2 (BMP-2) at a concentration that is about 10% of the concentration of BMP-2 needed for optimal osteoinduction by BMP-2 in the absence of 6-Bnz-cAMP.

9. A method of tissue engineering comprising:
contacting pre-osteoblast cells on PLAGA scaffolds with an effective concentration of N⁶-Benzoyladenosine-3′,5′-cyclic monophosphate (6-Bnz-cAMP).

10. The method of claim 9, wherein the method bone tissue engineering is further a method of proliferation of osteoblasts, a method of proliferation of osteoblast-like cells, a method of bone grafting, a method of surgical bone repair, a method of bone replacement, a method of bone regeneration, or a combination thereof.

11. The method of claim 9, wherein the concentration of 6-Bnz-cAMP is a concentration sufficient to activate the Protein Kinase A pathway.

12. The method of claim 9, wherein the concentration of 6-Bnz-cAMP is about 100 μM.

13. The method of claim 9, wherein the concentration of 6-Bnz-cAMP is about 50 μM to about 200 μM.

14. The method of claim 9, further comprising contacting the pre-osteoblast cells, at the same time they are in contact with 6-Bnz-cAMP, with Bone Morphogenetic Protein 2 (BMP-2) at a concentration that is about 10% of the concentration of BMP-2 needed for optimal osteoinduction by BMP-2 in the absence of 6-Bnz-cAMP.

15. The method of claim 14, wherein the pre-osteoblast cells are mesenchymal stem cells.

16. A method of preparing osteoblasts or osteoblast-like cells for use in bone tissue engineering comprising:
contacting pre-osteoblast cells on PLAGA scaffolds with an effective concentration of N⁶-Benzoyladenosine-3′,5′-cyclic monophosphate (6-Bnz-cAMP);
promoting initial cell adhesion and cell proliferation of such cells, and
thereby inducing differentiation of cells to osteoblasts or osteoblast-like cells on PLAGA scaffolds.

17. The method of claim 16, wherein the pre-osteoblast cells are mesenchymal stem cells.

18. A method of using the osteoblasts or osteoblast-like cells prepared by the method of claim 16 in vivo, in a method of treatment chosen from bone grafting, surgical bone repair, bone replacement, bone regeneration, or a combination thereof.
